(19) United States
(12) Patent Application Publication
(10) Pub. No.: US 2004/0171934 A1
(43) Pub. Date: Sep. 2, 2004

(54) Magnetic resonance system with multiple independent tracking coils

(76) Inventors: I. John Khan, Bridgewater, NJ (US); Carl O. Buyer, Phillipsburg, NJ (US); Charles L. Dumoulin, Ballston Lake, NY (US); Robert D. Darrow, Scotia, NY (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/773,513

(22) Filed: Feb. 6, 2004

Related U.S. Application Data
(60) Provisional application No. 60/445,531, filed on Feb. 6, 2003.

(51) Int. Cl. A61B 5/05; A61B 6/00; A61M 25/00; G01V 3/00

(52) U.S. Cl. 600/435; 600/422

(57) ABSTRACT

A magnetic resonance system has been developed for actively tracking the three-dimensional positions of numerous coils provided on one or more medical devices. One particular example of a novel magnetic resonance system of the present invention is capable of simultaneously tracking the positions of up to 32 coils or more, which may be provided on the medical device(s). As an example, catheter devices having a large number of independent tracking coils have been constructed, in which each coil has a direct connection to one at least the same number of receivers in the magnetic resonance system. Accordingly, physicians can obtain real-time visualization of the positions of medical devices using a magnetic resonance system, with sufficient frame-rates to guide the manipulation of the medical devices within the body of a patient. The medical devices may include catheters and guidewires. The magnetic resonance tracking system can track multiple devices simultaneously, as long as the total number of tracking coils on the medical devices does not exceed the total number of receivers in the magnetic resonance system.

### CROSS-REFERENCE TO RELATED APPLICATION

This application claims priority of U.S. Provisional Patent Application No. 60/445,531, filed Feb. 6, 2003.

### BACKGROUND AND SUMMARY OF THE INVENTION

1. Technical Background

The present invention generally relates to magnetic resonance systems for actively tracking medical devices having numerous tracking coils, and methods relating to the system.

2. Discussion

A magnetic resonance system has been developed for actively tracking the three-dimensional positions of numerous coils provided on a medical device. Such a medical device may be a catheter or any other suitable medical device, including guidewires, etc.

One particular example of a novel magnetic resonance system of the present invention is capable of simultaneously tracking the positions of multiple tracking, which may be provided on one or more medical devices. As an example, catheter devices having a large number of independent tracking coils have been constructed, in which each coil has a direct connection to one of at least the same number of receivers in the magnetic resonance system.

A method of using a magnetic resonance tracking system according to the present invention employs a non-selective radiofrequency pulse that excites all spins within the imaging volume of the magnet. Magnetic resonance signals from each coil are detected in the presence of magnetic field gradients and processed to determine the three-dimensional coordinates of each coil. The three-dimensional structure of the device can then be presented as a graphic overlay on a previously acquired magnetic resonance image.

During prior percutaneous medical procedures, various medical devices are often manipulated within a patient under image guidance using an X-ray video system called fluoroscopy. These medical devices can include flexible structures such as guidewires and catheters that are inserted into delicate vascular structures. In X-ray fluoroscopy, real-time visualization of some length of a medical device may be desirable for proper placement of the medical device.

Accordingly, it would be desirable to provide physicians with real-time visualization of medical devices using a magnetic resonance system. It may also be desirable to obtain sufficient frame-rates and visualization along the length of the medical device to guide its manipulation within the body of a patient.

One embodiment of this concept was constructed to evaluate the real-time visualization and utility of diagnostic or interventional magnetic resonance catheters employing a large number of active tracking coils. In this embodiment, thirty-two independent tracking coils were provided along the body of a catheter.

The display defines a scan plane and superimposes the positions of each coil in the scan plane onto a corresponding position on a previously recorded image. The display also depicts some type of visible indicator connecting adjacent coils on the medical device.

Active magnetic resonance device tracking methods follow medical devices with small magnetic resonance receiving coils incorporated into the device. Transverse spin magnetization is generated over the volume of interest by a large transmit coil (typically the body coil) with a non-selective radiofrequency pulse. Magnetic resonance signals are detected in the presence of an applied magnetic field gradient, but only those signals near the small tracking coils are detected. The magnetic resonance signals are then processed to determine the X, Y and Z coordinates of the coil.

The present invention may also incorporate multiple tracking coils provided on multiple medical devices. In other words, the magnetic resonance system may track the positions of the various coils, and indicates the positions of the coils and their corresponding medical device, for each of multiple medical devices.

These and other possible objects, features and advantages of the present invention will be apparent from and clearly understood through a consideration of the following detailed description of the preferred embodiment.

### BRIEF DESCRIPTION OF THE DRAWINGS

In the course of this description of preferred embodiments, reference will be made to the attached drawings, wherein:

FIG. 1 is a partial elevation view of a magnetic resonance catheter having multiple tracking coils, arranged according to the principles of the present invention;

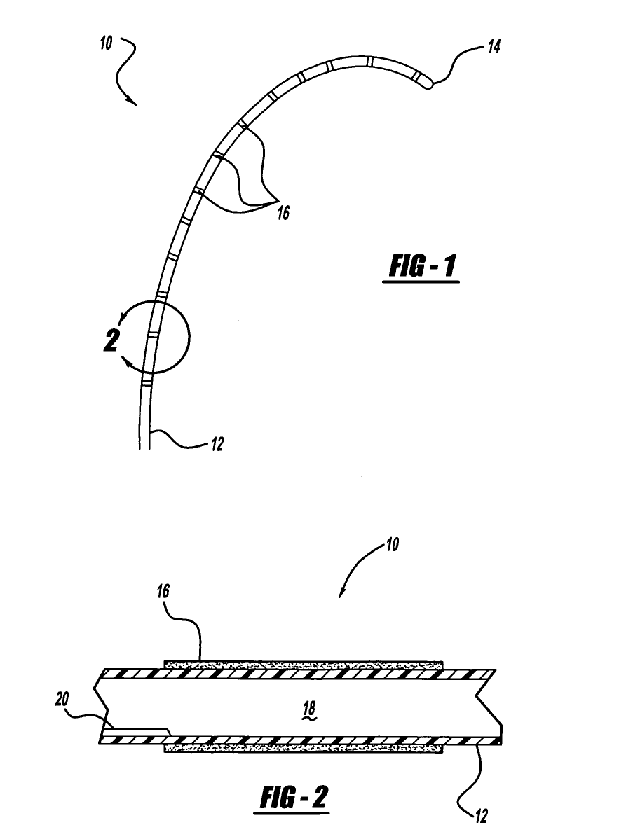

FIG. 2 is an enlarged partial cross-section view of a portion of the magnetic resonance catheter of FIG. 1; and

FIG. 3 is a depiction of a magnetic resonance catheter having multiple tracking coils and a test object, viewed using a magnetic resonance system.

### DESCRIPTION OF THE PREFERRED EMBODIMENTS

The following description of the preferred embodiments of the present invention is merely illustrative in nature, and as such it does not limit in any way the present invention, its application, or uses. Numerous modifications may be made by those skilled in the art without departing from the true spirit and scope of the invention.

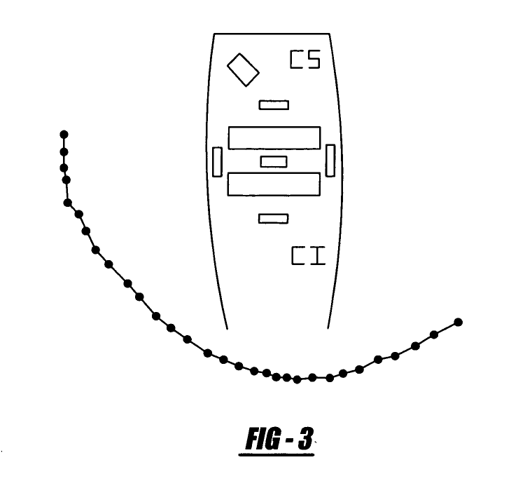

One possible arrangement of a magnetic resonance system according to the present invention is shown in the drawings. Catheter system 10 includes a catheter shaft 12 having a distal end 14, and a plurality of tracking coils 16. The catheter shaft may define a passage or lumen 18, and each coil 16 is coupled with the magnetic resonance system by a wire 20. A cross-section of a portion of the catheter system 10 is depicted in FIG. 2, in which coil 16 is shown in a diagrammatic fashion.

A patient is shown in FIG. 4 on a support table 22 in a homogeneous magnetic field generated by a magnet 26 in a magnet housing 24. Magnet 26 and magnet housing 24 are cylindrical, and are shown sectioned in half for clarity purposes. A medical device 32, which in this example is shown as a catheter, is inserted into the patient. The patient is surrounded by cylindrical magnetic field gradient coils 28, which create magnetic field gradients of predetermined strength at predetermined times. Gradient coils 28 generate magnetic field gradients in three mutually perpendicular directions. An external coil 30 also surrounds the patient, which emits radiofrequency energy at predetermined times at the predetermined frequency.

Medical device 32 is inserted into the patient by a physician 34. The particular medical device may be a guidewire, a catheter, an endoscope, a laparoscope, a biopsy needle, surgical implement, therapy delivery implement or other similar medical device. Medical device 32 may be tracked according to the method disclosed in the U.S. Pat. Nos. 5,307,808 and 5,715,822 to Dumoulin et al. and Watkins et al., the disclosures of which are incorporated herein by reference.

The medical device contains a series of radiofrequency coils which detect magnetic resonance signals generated in the patient, responsive to the radiofrequency field created by external coil 30. Since the radiofrequency coils are small, the regions of sensitivity are also small. Consequently, the detected signals have Lannor frequencies which arise only from the strength of the magnetic field in the immediate vicinity of the coil.

These detected signals are sent to an imaging and tracking unit 36 where they are analyzed. The positions of the series of radiofrequency coils are determined in the imaging and tracking unit 36, and are displayed on a display 38. The positions of the radiofrequency coils are displayed on the display 38 by superimposing them as a graphic symbol on a conventional magnetic resonance image.

In alternative embodiments of the invention, the graphic symbols representing the medical device and its coils are superimposed on diagnostic images obtained with other imaging systems, such as a computed tomography (CT) scanner, a Positron Emission Tomography system, or an ultrasound scanner. Other embodiments of the present invention display the position of the device numerically or as a graphic symbol without reference to a diagnostic image.

The magnetic resonance tracking systems of the present invention can track multiple devices simultaneously, as long as the total number of tracking coils on the medical devices does not exceed the total number of receivers in the magnetic resonance system. The method can acquire full three-dimensional coordinates for each coil at frame rates up to twenty-four positions per second with minimal or no latency.

Also, the method is acoustically quiet because the system is scanning the point positions of the several coils, rather than scanning an entire magnetic resonance image. Real-time manipulation of the device can thus be performed without hearing protection.

Furthermore, since the medical device incorporates discrete coils along its length, its entire length can be followed, even when the catheter is not in the scan plane. The systems of the present invention are thus also capable of presenting the positions of the medical device(s) on more than one magnetic resonance image, in other words, biplane tracking.

### EXAMPLE

In a particular example of a configuration selected from among various possible arrangements of the present invention, a catheter has been constructed with thirty-two magnetic resonance tracking coils, yet having a relatively small size or diameter of 8 French size. Each coil on this particular example catheter was made with ten turns of 31 gauge copper wire, and was connected to a distribution port with a 31 gauge coaxial cable. The coils were spaced at an interval of 1.5 centimeters. The example catheter had an inner working lumen suitable for receiving a 0.014 inch guidewire.

The example magnetic resonance scanning system was capable of tracking up to thirty-two magnetic resonance coils in real-time. The magnetic resonance system had thirty-two receive channels that were phase-locked with the transmitter to a single frequency source. Each receiver was connected to an imaging receive coil or to an alternate input port which can be connected to a single tracking coil. The example magnetic resonance tracking system employed a Hadamard multiplexing algorithm to minimize artifacts that may result from local variations in magnetic susceptibility.

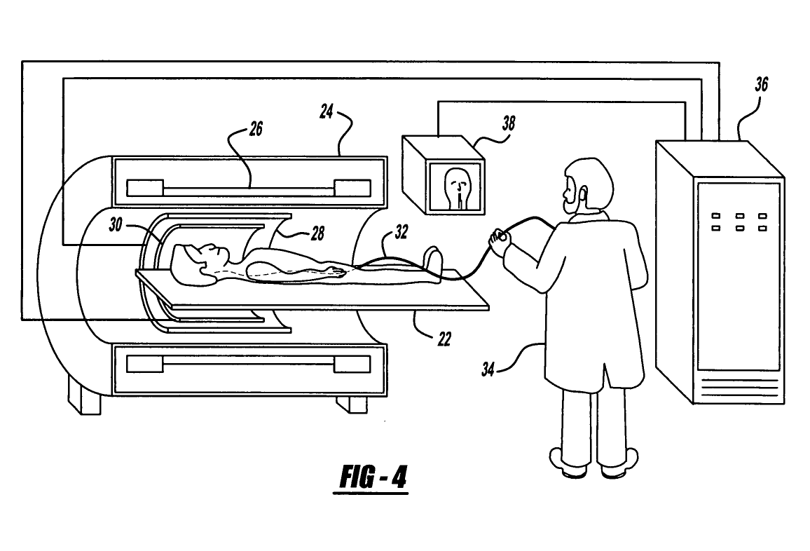

FIG. 3 illustrates one way in which the example information from multiple coils of the tracking system can be presented to the operator or physician. In this example, the example catheter was laid near a magnetic resonance phantom below the imaging plane. A coronal gradient-echo image of the phantom was then acquired. Magnetic resonance tracking was performed and the device manipulated in real-time. The position of each coil was presented to the operator as a green dot superimposed upon the magnetic resonance image on an in-room display. Adjacent coils were depicted by the system software as being connected with a line.

Numerous arrangements of and modifications to the present invention will be readily apparent to those skilled in the art. For example, a separate guidewire lumen and a distal guidewire port may be provided in the catheter shaft to allow the retrieval system to be inserted over a guidewire. Also, the distal end of the catheter shaft may be pre-curved or shaped, and/or may be steerable by means of a separate wire or filament to improve the alignment of the retrieved filter to the opening of the catheter shaft. Also, the shaft or main wire loop may be provided with radiopaque marker bands, which are visible on a fluoroscope or X-ray video screen.

It will be understood that the embodiments of the present invention which have been described are illustrative of some of the applications of the principles of the present invention. Various modifications may be made by those skilled in the art without departing from the true spirit and scope of the invention.

1. A magnetic resonance system for therapeutically treating a patient, comprising:
a medical device adapted to be inserted into a patient, having three or more magnetic resonance tracking coils;
a magnetic resonance system having a number of receivers equal to or greater than the number of coils, a tracking system coupled with each of the receivers to independently track the three-dimensional positions of all of the coils at a rate of at least about 5 positions per second;
a display depicting a magnetic resonance image defining a scan plane and superimposing the position of each coil in the scan plane onto a corresponding position on the magnetic resonance image; the display also depicting a visible indicator connecting adjacent coils on the medical device.

2. The magnetic resonance system in accordance with claim 1, further comprising one or more additional medical devices, each having at least one magnetic resonance tracking coil; each magnetic resonance tracking coil being coupled with a corresponding receiver of the magnetic resonance system.

3. The magnetic resonance system in accordance with claim 1, wherein the medical device is provided with a multitude of twenty or more magnetic resonance tracking coils.

4. The magnetic resonance system in accordance with claim 1, wherein the medical device is a catheter.

5. The magnetic resonance system in accordance with claim 1, wherein the medical device is a guidewire.

6. The magnetic resonance system in accordance with claim 1, wherein the tracking system uses a Hadamard multiplexing algorithm to minimize artifacts.

7. The magnetic resonance system in accordance with claim 1, wherein the visible indicator is a line shown on the display, connecting the indicated positions of adjacent coils.

8. The magnetic resonance system in accordance with claim 1, wherein the magnetic resonance system scans only the positions of the coils and operates at a lower acoustic noise level than scanning to obtain an image.

9. The magnetic resonance system in accordance with claim 1, in which the tracking system tracks the positions of all of the coils, wherein at least some of the coils are outside the current scan plane.

10. The magnetic resonance system in accordance with claim 1, further comprising at least one additional magnetic resonance image defining a second scan plane, wherein the positions of each of the tracking coils in the second scan plane are superimposed onto a corresponding position on the additional magnetic resonance image.

11. A magnetic resonance system for therapeutically treating a patient, comprising:
at least two medical devices adapted to be inserted into a patient, each having at least three magnetic resonance tracking coils;
a magnetic resonance system having a number of receivers equal to or greater than the total number of coils, a tracking system coupled with each of the receivers to independently track the three-dimensional positions of all of the coils at a rate of at least about 5 positions per second;
a display depicting a magnetic resonance image defining a scan plane and superimposing the position of each coil in the scan plane onto a corresponding position on the magnetic resonance image; the display also depicting a visible indicator connecting adjacent coils on each of the medical devices.

12. A magnetic resonance system for therapeutically treating a patient, comprising:
a medical device adapted to be inserted into a patient, having three or more magnetic resonance tracking coils;
a magnetic resonance system having a number of receivers equal to or greater than the total number of coils, a tracking system coupled with each of the receivers to independently track the three-dimensional positions of all of the coils at a rate of at least about 5 positions per second;
a display depicting at least a first and second magnetic resonance image defining a first and second scan plane and superimposing the position of each coil in one of the scan planes onto a corresponding position on the corresponding magnetic resonance image; the display also depicting a visible indicator connecting adjacent coils on the medical device.
